(19) United States
(12) Patent Application Publication
(10) Pub. No.: US 2024/0085428 A1
(43) Pub. Date: Mar. 14, 2024

(54) Assay for Detecting Collagen XI Biomarkers

(71) Applicant: Nordic Bioscience A/S, Herlev (DK)

(72) Inventors: Nicholas Willumsen, Dyssegard (DK); Neel Ingemann Nissen, Herlev (DK); Morten Asser Karsdal, København Ø (DK)

(73) Assignee: Nordic Bioscience A/S, Herlev (DK)

(21) Appl. No.: 18/268,694

(22) PCT Filed: Dec. 20, 2021
(86) PCT No.: PCT/EP2021/086770

§ 371 Date: Jun. 21, 2023

(30) Foreign Application Priority Data
Dec. 22, 2020 (GB) 2020399.8

(51) Int. Cl. G01N 33/574; C07K 16/32; G01N 33/68

(52) U.S. Cl. CPC G01N 33/57488; C07K 16/32; G01N 33/6887; G01N 2333/78

(57) ABSTRACT

Disclosed herein are monoclonal antibodies that specifically recognise and bind to a C-terminus of a peptide that is the C-terminus neo-epitope formed on cleavage of the N-terminal pro-peptide of the α1-chain of type XI collagen at A′511↓, and immunoassay methods and kits using and containing the antibodies. The immunoassay methods may be used for detecting and/or monitoring and/or assessing the severity or prognosis of diseases, such as, but not limited to, cancer.

### FIELD OF INVENTION

The present invention relates to an immunoassay for detecting neo-epitopes formed on cleavage of the N-terminal pro-peptide of the α1-chain of type XI collagen, and/or for quantitating the amount of said neo-epitopes in a sample. The present invention also relates to monoclonal antibodies that specifically bind to said neo-epitopes, and to kits comprising said antibodies for carrying out said immunoassays. The immunoassays, antibodies and kits may be used for detecting, monitoring and/or assessing the severity or prognosis of a disease, such as for example pancreatic cancer, chronic pancreatitis, a melanoma, bladder cancer, breast cancer, colorectal cancer, head and neck cancer, kidney cancer, liver cancer, lung cancer, ovarian cancer, prostate cancer, or stomach cancer, in a patient.

### BACKGROUND

Pancreatic cancer (PC) is only the 14th most prevalent cancer, yet it still accounts for 4.5% of all cancer deaths worldwide (World Health Organization: Latest global cancer data, 2018; Bray et al., 2018). A major reason to this, is that most patients present with metastasis at the time of diagnosis, leading to only 10% of the patients undergoing curative surgery. Other standard of care treatment-options include different chemotherapy regiments, however these drugs are mainly used in the palliative setting (McGuigan et al., 2018).

Pancreatic ductal adenocarcinoma (PDAC) is characterized by severe tumor fibrosis/desmoplasia resulting in an avascular and hypoxic tumor microenvironment (TME). The fibrotic TME in PC is often characterized by an extensive number of cancer-associated fibroblasts (CAFs) which are fibroblasts that are activated due to persistent injurious stimuli from the surrounding stroma (Hosein, Brekken and Maitra, 2020). CAFs are the most abundant cell type in the tumor microenvironment and known to dictate tumor outcome (Franco et al., 2010). CAFs secrete growth factors, enzymes and extracellular matrix (ECM) molecules that promote tumor growth, angiogenesis, tumor invasion and metastasis and they are therefore thought of as a rich reservoir of tumor-promoting factors (Kalluri and Zeisberg, 2006). Recognizing this important role of CAFs and a desmoplastic stroma in tumor development and progression, identifying and targeting stromal components of the tumor is a field of extensive research in cancer. In particular, novel non-invasive biomarkers that could provide additional insight into the tumor biology in patients with a stroma- and CAFs-rich TME, such as that found in the tumor of patients with PC, would be highly beneficial.

Biomarkers for predicting immune checkpoint inhibitor (ICI) efficacy, and thereby allowing for patient selection for current ICI regimens and new combination therapies, would likewise be very beneficial. Patients with T-cell excluded tumors have poor effect on ICI therapy. T-cell exclusion is driven by upregulated TGF-β-signaling and activated fibroblasts, which produce increased levels of fibrillar collagens (desmoplasia).

The main ECM-proteins secreted by CAFs are collagens. The fibroblast derived collagens include collagen I, II, III, V, IV and XI. These collagens are the main constituents of the tumor stromal compartment and are located in the interstitial matrix. While the basement membrane collagens, e.g. collagen IV, and their role in cancer is well described, much less is known about the fibroblast derived collagens. However, evidence has shown that these collagens might play a major role in tumor initiation and progression (Nissen, Karsdal and Willumsen, 2019). Biomarkers quantifying specific fibroblast-derived collagen fragments have shown to predict patient outcome in various cancer diseases (Willumsen et al., 2013, 2014; Kang et al., 2014; Bager et al., 2015; Kehlet et al., 2016; Jensen et al., 2018). Recently, it has been shown that PRO-C3, a biomarker targeting the pro-peptide of collagen III, was prognostic for overall survival (OS) in PC (Chen et al., 2020). In addition, in another study it was shown that PRO-C3 was able to predict patients responding to a novel anti-fibrotic drug PEGPH20 (Wang et al., 2018).

Several studies have focused on identifying CAF-specific genes/proteins that could serve as novel biomarkers. Interestingly, one of the most specific CAF genes that have been identified so far is COL11A1 which encodes the α1-chain of type XI collagen (Vázquez-Villa et al., 2015). Type XI collagen is a minor fibrillar collagen expressed by chondrocytes, osteoclasts, CAFs, but not quiescent fibroblasts (García-Pravia et al., 2013; Raglow and Thomas, 2015; Vázquez-Villa et al., 2015). The function of type XI collagen has been suggested to involve maintaining of proper fibril formation and diameter. Mature type XI collagen is a heterotrimer consisting of an α1-, α2- and α3-chain, which are synthesized as pro-collagens and where the pro-peptides are subsequently proteolytically cleaved to yield mature type XI collagen. The N-terminal pro-peptide of the α1-chain of type XI collagen has been shown to be only partly released in vitro by BMP-1 cleavage at A′253↓ (Rousseau et al., 1996). However, the N-terminal pro-peptide of the α1-chain of type XI collagen has also been shown to be cleaved at the very end of the pro-peptide by N-proteinase cleavage at A′511↓ (Yoshioka and Ramirez, 1990).

U.S. Pat. No. 9,702,879 B2 (Serra et al) describes an in vitro method for detecting the presence of an invasive carcinoma in an individual, based on detecting the amount of proCOL11A1 (the pro-collagen of the α1-chain of type XI collagen) in a sample taken from said individual. The method uses a monoclonal antibody that binds to one or more epitopes present in a hydrophilic domain located in the N-terminal region of proCOL11A1, consisting of amino acids 268 to 400 of prCOL11A1. The authors of this document concluded that this region would be the most promising region for generating a monoclonal antibody specific to proCOL11A1, due to its hydrophobicity (indicating that the region is exposed in the protein's native conformation) and due to it containing the sequence of lowest homology with other collagen isoforms of the greatest similarity with proCOL11A1.

WO2020/065078A1 (Willumsen et al) describes the generation of a monoclonal antibodies targeting a C-terminus neo-epitope formed on cleavage of the N-terminal pro-peptide of the α1-chain of type XI collagen at A′253↓ and the use of said antibodies in a competitive ECLIA (Electro-ChemiLuminescence ImmunoAssay) for diagnosing and/or monitoring non-small cell lung cancer in a patient.

Nevertheless, there remains a need for novel, non-invasive biomarkers that may be used clinically to identify patients suffering from cancers, such as but not limited to cancers with stroma- and CAFs-rich TME (such as are found in the tumors of patients with pancreatic cancer) and cancers with T-cell excluded tumors that may be predictive of response and survival outcomes for treatment with ICI therapy, and other diseases characterised by or exhibiting severe fibrosis.

### SUMMARY

The applicant has now developed an enzyme-linked immunosorbent assay (ELISA) targeting the C-terminus neo-epitope generated by enzymatic cleavage of the N-terminal pro-peptide of the α1-chain of type XI collagen at A′511↓, and has demonstrated the prognostic value of this biomarker in patients with PC, patients with chronic pancreatitis (CP), and patients with metastatic melanoma and patients with various other types of cancer.

Accordingly, in a first aspect the application provides a monoclonal antibody that specifically recognises and binds to a C-terminus of a peptide having the C-terminus amino acid sequence DGSKGPTISA (SEQ ID NO: 1) (which comprises the C-terminus neo-epitope formed on cleavage of the N-terminal pro-peptide of the α1-chain of type XI collagen at A′511↓). The amino acid sequence DGSKGPTISA (SEQ ID NO: 1) is also referred to herein as the “PRO-C11-511 target sequence”, and peptides consisting of said sequence or having said sequence as their C-terminus are also referred to herein as the “PRO-C11-511 target peptide”.

As used herein the term “C-terminus” refers to a C-terminal peptide sequence at the extremity of a polypeptide, i.e. at the C-terminal end of the polypeptide, and is not to be construed as meaning in the general direction thereof.

As used herein, the terms “peptide” and “polypeptide” are used synonymously.

As used herein, the term “neo-epitope” refers to an epitope that is formed on cleavage of a peptide, but that is not present, or recognisable by antibodies, in the non-cleaved peptide (for example because the free —NH₂ or —COOH group at the cleavage site forms part of the epitope that is recognised and specifically bound to by the antibody).

In preferred embodiments, the monoclonal antibody does not specifically recognise or bind to a peptide having the C-terminus amino acid sequence DGSKGPTISAQ (SEQ ID NO: 2) (i.e. the PRO-C11-511 target sequence extended at its C-terminus by the addition of a glutamine residue).

In preferred embodiments, the monoclonal antibody does not specifically recognise or bind to a peptide having the C-terminus amino acid sequence DGSKGPTIS (SEQ ID NO: 3) (i.e. the PRO-C11-511 target sequence truncated by removal of the final alanine residue).

Preferably, the monoclonal antibody is a monoclonal antibody that is raised against a synthetic peptide having the C-terminus amino acid sequence DGSKGPTISA (SEQ ID NO: 1).

As used herein the term “monoclonal antibody” refers to both whole antibodies and to fragments thereof that retain the binding specificity of the whole antibody, such as for example a Fab fragment, F(ab′)2 fragment, single chain Fv fragment, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain contain the variable region, while the C-terminal portions of each of the heavy and light chains make up the constant region. The variable region comprises three complementarity determining regions (CDRs), which are primarily responsible for antigen recognition. The constant region allows the antibody to recruit cells and molecules of the immune system. Antibody fragments retaining binding specificity comprise at least the CDRs and sufficient parts of the rest of the variable region to retain said binding specificity.

In the methods of the present invention, a monoclonal antibody comprising any constant region known in the art can be used. Human constant light chains are classified as kappa and lambda light chains. Heavy constant chains are classified as mu, delta, gamma, alpha, or epsilon, and define the antibody's isotype as IgM, IgD, IgG, IgA, and IgE, respectively. The IgG isotype has several subclasses, including, but not limited to IgGI, IgG2, IgG3, and IgG4. The monoclonal antibody may preferably be of the IgG isotype, including any one of IgGI, IgG2, IgG3 or IgG4.

The CDR of an antibody can be determined using methods known in the art such as that described by Kabat et al. Antibodies can be generated from B cell clones as described in the examples. The isotype of the antibody can be determined by ELISA specific for human IgM, IgG or IgA isotype, or human IgG1, IgG2, IgG3 or IgG4 subclasses. The amino acid sequence of the antibodies generated can be determined using standard techniques. For example, RNA can be isolated from the cells, and used to generate cDNA by reverse transcription. The cDNA is then subjected to PCR using primers which amplify the heavy and light chains of the antibody. For example primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lamda chain together with primers which anneal to the V kappa or V lambda leader sequence. The full length heavy and light chains can be generated and sequenced.

In a second aspect, the application provides a method of immunoassay, the method comprising:
(i) contacting a biofluid sample from a patient with a monoclonal antibody according to the first aspect; and
(ii) detecting and determining the amount of binding between said monoclonal antibody and peptides in the sample.

In a preferred embodiment, the method is a method of immunoassay for detecting and/or monitoring a disease in a patient and/or assessing the severity or prognosis of a disease in a patient, the method further comprising:
(iii) correlating said amount of binding of said monoclonal antibody as determined in step (ii) with values associated with normal healthy subjects, and/or with values associated with known disease severity or prognosis, and/or with values obtained from said patient at a previous time point, and/or with a predetermined cut-off value.

In some embodiments, the disease is a cancer, such as but not limited to pancreatic cancer, melanoma, bladder cancer, breast cancer, colorectal cancer, head and neck cancer, kidney cancer, liver cancer, lung cancer, ovarian cancer, prostate cancer, or stomach cancer. The cancer may be a cancer with a stroma- and cancer associated fibroblast-rich tumor micro environment. The cancer may in particular be pancreatic ductal adenocarcinoma or metastatic melanoma.

In some embodiments, the disease is a pancreatic disease, such as but not limited to pancreatic cancer (particularly pancreatic ductal adenocarcinoma) or chronic pancreatitis.

Where the method is a method for assessing the severity or prognosis of a disease, the method may for example be for assessing the stage of a cancer; or for assessing a likely period of patient survival or progression-free survival; or for assessing a likely response to a medical intervention, such as a likely period of patient survival or progression-free survival with treatment with one or more drugs (such as one or more chemotherapeutic agents (i.e. cytotoxic agents) and/or immune checkpoint inhibitors).

The sample is preferably a biofluid. The biofluid may be, but is not limited to, blood, serum, plasma, urine or a supernatant from cell or tissue cultures. Preferably the biofluid is blood, serum or plasma.

The immunoassay may be, but is not limited to, a competition assay or a sandwich assay. The immunoassay may, for example, be a radioimmunoassay or an enzyme-linked immunosorbent assay (ELISA). Such assays are techniques known to the person skilled in the art.

As used herein the term “amount of binding” refers to the quantification of binding between the monoclonal antibody and peptides in the patient sample. Said quantification may for example be determined by comparing the measured values of binding in the patient sample against a calibration curve produced using measured values of binding in standard samples containing known concentrations of a peptide to which the antibody specifically binds, in order to thereby determine the quantity of peptide to which the antibody specifically binds in the patient sample. In the Examples set out below, an ELISA method is used in which spectrophotometric analysis is used to measure the amount of binding both in the patient samples and when producing the calibration curve. However, any suitable analytical method can be used.

As used herein the term “predetermined cut-off value” means an amount of binding that is determined statistically to be indicative of a high likelihood of a disease or a particular severity thereof (or prognosis therefor) in a patient, in that a measured value of the target peptide in a patient sample that is at or above the statistical cut-off value corresponds to at least a 70% probability, preferably at least an 75% probability, more preferably at least an 80% probability, more preferably at least an 85% probability, more preferably at least a 90% probability, and most preferably at least a 95% probability of the presence of said disease or said particular severity thereof (or prognosis therefor).

As used herein, the term “values associated with normal healthy subjects” means standardised quantities of binding determined by the method described supra for samples from subjects considered to be healthy, i.e. without disease; and the term “values associated with known disease severity or prognosis” means standardised quantities of binding determined by the method described supra for samples from patients known to have disease of a known severity or prognosis.

In a third aspect, the application relates to an immunoassay kit comprising a monoclonal antibody according to the first aspect, and at least one of;
a streptavidin coated well plate;
a biotinylated peptide Biotin-L-DGSKGPTISA (SEQ ID NO: 4), wherein L is an optional linker;
a secondary antibody for use in a sandwich immunoassay;
a calibrator protein comprising the sequence DGSKGPTISA (SEQ ID NO: 1);
an antibody biotinylation kit;
an antibody HRP labelling kit;
an antibody radiolabelling kit; and
an assay visualisation kit.

### FIGURES

FIG. 1: Schematic illustration of collagen type XI with descriptions of the PRO-C11-253 (A) and PRO-C11-511 (B) biomarker targets.

FIG. 2: Specificity test of the PRO-C11-253 and PRO-0511 assays. A) The reactivity of the PRO-C11-253 antibody was tested against the standard (STD), elongated, truncated and non-sense (non-sense STD) peptides and a non-sense coater. It was also tested against three different de-selection peptides. B) The reactivity of the PRO-C11-511 antibody was tested against the standard (STD), elongated, truncated and non-sense (non-sense STD) peptides, a non-sense coater and a de-selection peptide. % B/B0: B equals the OD at x ng/ml peptide and BO equals the OD at 0 ng/ml peptide.

FIG. 3: Individual biomarker measurement in healthy controls (n=20), and in chronic pancreatitis (n=12) and pancreatic cancer (n=39) patients. Black line is showing the median. A) PRO-C11-253, B) PRO-C11-511. Ns: non-significant. * p<0.05. **** p<0.0001.

FIG. 4: Kaplan Meier survival plots showing the association between high (>75%) and low (<75%) biomarker levels of PRO-C11-253, PRO-C11-511 and overall survival. A) PRO-C11-253, B) PRO-C11-511

FIG. 5: Individual PRO-C11-511 biomarker measurement in pancreatic cancer (n=686). Black line is showing the median. * p<0.05. *** p<0.001.

FIG. 6: A) Kaplan Meier plots showing association between high (>75%) and low (>75%) levels of PRO-C11-511 and overall survival in pancreatic cancer (n=686). B) Plot showing percent of patients alive 2-years post baseline for high (>75%) and low (>75%) biomarker measurements.

FIG. 7: Kaplan Meier plot for evaluating progression-free survival and overall survival associated with PRO-C11-511 at baseline by grouping (dichotomizing) at the 75th percentile (Q1+Q2+Q3 vs Q4) in pembrolizumab treated metastatic melanoma patients.

FIG. 8: PRO-C11-511 was measured in the serum of multiple cancer patient groups and healthy controls. PRO-C11-511 levels are presented as Tukey-style boxplots with data-point jitter: horizontal bars indicate the median; upper- and lower hinges of the box indicate the first and third quartiles (the 25th and 75th percentiles); whiskers extend from the hinges to the largest or smallest value but no further than 1.5*IQR (where IQR is the inter-quartile range between the first and third quartiles) in either the positive or negative direction. Samples measuring below the lower limit of measurement range (LLMR) were given the value of LLMR, as determined in the validation of PRO-C11-511. **** indicates a p-value below 0.0001. *** below 0.001. ** below 0.01. * below 0.05.

### EXAMPLES

The presently disclosed embodiments are described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Material and Methods

### Collagen XI Assay Development—Identification of Target Sequences and Monoclonal Antibody Production

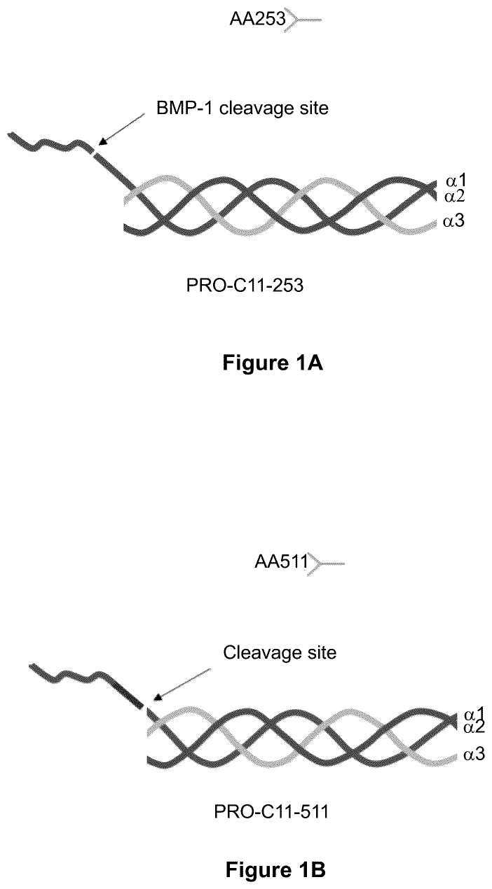

The N-terminal pro-peptide of the α1-chain of type XI collagen has been shown to be cleaved at either amino acid A′253↓ or amino acid (Rousseau et al., 1996). A′511↓ (Yoshioka and Ramirez, 1990). To evaluate the relevance of targeting these two sites monoclonal antibodies where raised against either the peptide ₂₄₄DSSAPKAAQA₂₅₃ (SEQ ID NO: 5) (which target sequence and peptide is referred to herein as PRO-C11-253) or ₅₀₂DGSKGPTISA₅₁₁ (SEQ ID NO: 1) (which target sequence and peptide is referred to herein as PRO-C11-511) (FIG. 1). The amino acid sequences were blasted for homology to other human secreted extracellular matrix proteins using NPS@: Network Protein Sequence Analysis with the UniprotKB/Swiss-prot database and found to be unique (Combet et al., 2000).

Immunogenic peptides (for PRO-C11-253: KLH-CGG-DSSAPKAAQA (SEQ ID NO: 6) and for PRO-C11-511: KLH-CGG-DGSKGPTISA (SEQ ID NO: 7)) were generated by covalently cross-linking the target peptide to Keyhole Limpet Hemocyanin (KLH) carrier protein using sulfosuccinimidyl 4-(N-maleimidomethyl) cyclohexane-1-carboxylate, SMCC (Thermo Scientific, Waltham, Mass., USA, cat. no. 22322). Glycine and cysteine residues were added at the N-terminal end to ensure right linking of the carrier protein. Monoclonal antibodies were generated by subcutaneous immunization of six-week-old Balb/C mice with 200 μL emulsified antigen containing 100 μg immunogenic peptide mixed with Specol (Invitrogen cat. no. 7925000) for the PRO-C11-253 immunogenic peptide and Sigma adjuvant system for the PRO-C11-511 immunogenic peptide (Sigma cat. no. S6322). Consecutive immunizations were performed at 2-week intervals until stable sera titer levels were reached. The mouse with the highest titer rested for four weeks and was then boosted with 100 μg immunogenic peptide in 100 μL 0.9% NaCl solution intravenously. Hybridoma cells were produced by fusing spleen cells with SP2/0 myeloma cells as previously described (Gefter, Margulies and Scharff, 1977). The resultant hybridoma cells were then cultured in 96-well microtiter plates and standard limited dilution was used to secure monoclonal growth.

The monoclonal antibodies were purified using protein-G-columns according to the manufacturer's instructions (GE Healthcare Life Sciences, Little Chalfont, UK, cat. #17-0404-01). The best antibody clone for each biomarker was selected based on a preliminary competitive ELISA for the reactivity towards the selection peptide (the target peptide, also referred to as the standard peptide), and not an elongated selection peptide with an additional amino acid added to the C-terminus of the target peptide sequence, a truncated selection peptide with a removal of the first C-terminus amino acid, a non-sense selection peptide and a non-sense biotinylated coating peptide (see table 1). A biotinylated selection peptide was used as a coating peptide (table 1). To screen for any potential cross-reactivity to other proteins and further test the antibody specificity, three peptides (derived from cystatin-M, Lysyl oxidase homolog 1 and Keratin-like protein KRT222) with one amino acid mismatch compared to the first six amino acids in the target sequence were included for PRO-C11-253 and one peptide (derived from mucin 4) for PRO-C11-511 (table 1).

TABLE 1
Synthetic peptides used for development and validation of the PRO-C11-253
and PRO-C11-511 ELISA assays

| | PRO-C11-253 | PRO-C11-511 |
|---|---|---|
| Peptide name | (amino acid sequence) | (amino acid sequence) |
| Standard peptide | DSSAPKAAQA↓ | DGSKGPTISA↓ |
| | (SEQ ID NO: 5) | (SEQ ID NO: 1) |
| Biotinylated coating | Biotin-DSSAPKAAQA | Biotin-DGSKGPTISA |
| peptide | (SEQ ID NO: 8) | (SEQ ID NO: 4) |
| Elongated peptide | DSSAPKAAQAQ | DGSKGPTISAQ |
| | (SEQ ID NO: 9) | (SEQ ID NO: 2) |
| Truncated peptide | DSSAPKAAQ | DGSKGPTIS |
| | (SEQ ID NO: 10) | (SEQ ID NO: 3) |
| Non-sense standard | LLARDFEKNY | DSSAPKAAQA |
| peptide | (SEQ ID NO: 11) | (SEQ ID NO: 5) |
| Non-sense coater peptide | LLARDFEKNY-K-biotin | Biotin-DSSAPKAAQA |
| | (SEQ ID NO: 12) | (SEQ ID NO: 8) |
| De-selection peptides: | DPQVQKAAQA (Cystatin-M, | VPQDAPTISA |
| | CYS-M) (SEQ ID NO: 13) | (mucin-4) |
| | PDPGPEAAQA (Lysyl oxidase | (SEQ ID NO: 16) |
| | homolog 1, LOXH-1) | |
| | (SEQ ID NO: 14) | |
| | DEEALKAAQA (Keratin-like | |
| | protein KRT222, KRT222) | |
| | (SEQ ID NO: 15) | |

### PRO-C11-253 and PRO-C11-253 ELISA Protocols

The competitive PRO-C11-253 and PRO-C11-511 ELISA assays were performed as follows, after determination of optimal ratio of antibody/coater peptide, incubation buffer, -time and -temperature, as well as conjugation of horseradish peroxidase to the PRO-C11-253 antibody (to increase the sensitivity of the PRO-C11-253 assay):

### PRO-C11-253 ELISA

A 96-well streptavidin-coated microplate plate was coated with 100 μL biotinylated coating peptide (0.5 ng/mL) dissolved in assay buffer (50 mM PBS-BTB 8 g NaCl/L, pH 7.4) for 30 min at 20° C. in darkness with shaking (300 rpm). After five times of washing (20 mmol/L TRIS, 50 mmol/L NaCl, pH 7.2) 20 μL of standard peptide or serum sample (pre-diluted 1:2) were added to appropriate wells, followed by the addition of 100 μL of the horseradish peroxidase (HRP)-conjugated antibody for PRO-C11-253 (25 ng/mL) dissolved in assay buffer and incubated for 20 hours at 4° C. in darkness with shaking (300 rpm). After washing five times, 100 uL Tetramethylbezidine (TMB) (Kem-En-Tec Diagnostics, Taastrup, Denmark) was added to the plates and incubated in darkness for 15 min at 20° C. with shaking (300 rpm). The reaction was stopped by adding 100 μL of 1% sulfuric acid. Plates were analyzed in a VersaMax ELISA microplate reader at 450 nM with 650 nm as reference. A standard curve was plotted using a 4-parametric mathematical fit model, and data were analyzed using GraphPad Prism version 8 (GraphPad Software, Inc.).

### PRO-C11-511 ELISA

A 96-well streptavidin-coated microplate plate was coated with 100 μL biotinylated peptide (1.9 ng/mL) dissolved in assay buffer (25 mM PBS-BTB 2 NaCl/L, pH 7.4) for 30 min at 20° C. in darkness with shaking (300 rpm). After five times of washing (20 mmol/L TRIS, 50 mmol/L NaCl, pH 7.2) 20 μL of standard peptide or serum sample (pre-diluted 1:2) were added to appropriate wells, followed by the addition of the antibody for PRO-C11-511 (18.8 ng/mL) dissolved in assay buffer and incubated 20 hours at 4° C. in darkness with shaking (300 rpm). After washing five times, 130 ng/mL secondary goat anti-mouse HRP-conjugated IgG antibody (Thermo Scientific, Waltham, MA, USA) diluted in assay buffer was added to each well, and incubated in darkness with shaking (300 rpm) for one hour at 20° C. After washing five times, 100 uL Tetramethylbezidine (TMB) (Kem-En-Tec Diagnostics, Taastrup, Denmark) was added to the plates and incubated in darkness for 15 min at 20° C. with shaking (300 rpm). The reaction was stopped by adding 100 μL of 1% sulfuric acid. Plates were analyzed in a VersaMax ELISA microplate reader at 450 nM with 650 nm as reference. A standard curve was plotted using a 4-parametric mathematical fit model, and data were analyzed using GraphPad Prism version 8 (GraphPad Software, Inc.).

### Technical Evaluation of PRO-C11-253 and PRO-C11-511 ELISAs

The technical performance of the PRO-C11-253 and PRO-C11-511 ELISA assays were evaluated with the following validation tests: Lower Limit of Measuring Range (LLMR), Upper Limit of Measuring Range (ULMR), Inter- and intra-assay variation, linearity, accuracy (spiking), analyte stability (freeze/thaw and storage) and interference.

The analytical measurement range was defined as the concentrations from LLMR to ULMR (the linear part of the standard curve) estimated from ten independent runs. The inter- and intra-assay variation was determined by ten independent runs on different days using five quality control samples covering the detection range, with each run consisting of double determinations of the samples. The five control samples included three human serum samples and two samples with standard peptide in buffer. Intra-assay variation was calculated as the mean coefficient of variance (CV %) within plates and the inter-assay variation was calculated as the mean CV % between the ten individual runs analyzed on different days. Linearity (dilution recovery) was determined with 2-fold dilutions of three human serum samples and calculated as percentage recovery of the un-diluted samples. Accuracy (spiking recovery) was assessed by combining eight human serum samples of known concentration and spiking recovery was calculated as the measured PRO-C11-253 and PRO-C11-511 amount percentage recovery of the theoretical amount. Analyte stability was determined by the effect of repeated freeze/thaw and in relation to temperature storage. Three serum samples were thawed and frozen four times followed by PRO-C11-253 and PRO-C11-511 measurements. The freeze/thaw recovery was calculated with the first cycle as reference. A 48-hour study was performed to determine analyte stability at 4° C. and 20° C. using three human serum samples. The level of PRO-C11-253 and PRO-C11-511 was measured after 4 h, 24 h and 48 h of storage, and recovery was calculated with non-stressed samples stored at −20° C. as reference. Interference was determined by adding a low/high content of hemoglobin (2.5/5 mg/mL), lipemia/lipids (1.5/5 mg/mL) and biotin (30/90 ng/mL) to a serum sample of known concentration. Recovery percentage was calculated with the serum sample as reference.

Patients with Chronic Pancreatitis and Pancreatic Cancer

The two biomarkers PRO-C11-253 and PRO-C11-511 were analysed in pretreatment serum samples from a discovery cohort including patients with PC (stage I-IV) (n=39), chronic pancreatitis (CP) (n=12) and age and gender matched healthy controls (n=20). Furthermore, PRO-C11-511 was analysed in pretreatment serum samples from a validation cohort including 686 patients with PC (stage I-IV). Healthy controls were obtained from the commercial vendor Valley BioMedical (VA, USA) (see table 2 for patient demographics). Valley BioMedical had Appropriate Institutional Review Board/Independent Ethical Committee approved sample collection and all subjects filed informed consent. All PC and CP patients were from the Danish BIOPAC study “BIOmarkers in patients with Pancreatic Cancer” (NCT03311776). Patients were recruited from six Danish hospitals from December 2008 until September 2017. PC patients had histologically confirmed tumors. The PC patients were treated with various types of chemotherapy according to national guidelines (www.gicancer.dk). The study was carried out in accordance with the recommendations of the Danish Regional Committee on Health Research Ethics. The BIOPAC protocol was approved by the Danish Regional Committee on Health Research Ethics (VEK ref. KA-20060113) and the Data Protection Agency (j.nr. 2006-41-6848). All subjects gave written informed consent in accordance with the Declaration of Helsinki, version 8. Blood samples were obtained at the time of diagnosis or before operation. Samples were processed according to nationally approved standard operating procedures for blood (www.herlevhospital.dk/biopac.dk). Serum samples and clinical data from patients were collected prospectively. Serum sample were measured blinded. Clinical data included: age, gender, number of metastatic sites, liver metastasis, BMI, stage, diabetes, tobacco use, alcohol use, CA19-9 (median), performance status (PS), The Charlson age comorbidity index (CACI), overall survival (OS).

TABLE 2
Patient demographics and clinical profile.
A) Discovery cohort. B) Validation Cohort.
DHAR: Danish Health Authority recommendations,
CA19-9: carbohydrate antigen (U/mL).
A)

| Clinical variables (healthy controls) | Study population (n = 20) | |
|---|---|---|
| Age, (years) | | |
| Median (min, max) | 58 | (45-72) |
| Gender, n (%) | | |
| Male | 10 | (50%) |
| Female | 10 | (50%) |
| Clinical variables (pancreatitis) | Study population (n = 12) | |
| Age, (years) | | |
| Median (min, max) | 60 | (49-79) |
| Gender, n (%) | | |
| Male | 10 | (83%) |
| Female | 2 | (17%) |
| Clinical variables (pancreas cancer) | Study population (n = 39) | |
| Age, (years) | | |
| Median (min, max) | 68 | (52-79) |
| Gender, n (%) | | |
| Male | 20 | (51%) |
| Female | 19 | (49%) |
| Number of metastatic sites, n (%) | | |
| ≤1 site | 31 | (79%) |
| >1 site | 8 | (21%) |
| Liver metastasis | | |
| Only liver | 10 | (26%) |
| Other | 29 | (74%) |
| BMI | | |
| Median (min, max) | 23 | (16-31) |
| Stage | | |
| 1 | 3 | (8%) |
| 2 | 16 | (41%) |
| 3 | 0 | |
| 4 | 20 | (51%) |
| Diabetes | | |
| Yes | 8 | (21%) |
| No | 31 | (79%) |
| Tobacco | | |
| Ever | 26 | (67%) |
| Never | 13 | (33%) |
| Alcohol | | |
| <DHAR | 31 | (79%) |
| >DHAR | 7 | (18%) |
| Unknown | 1 | (<1%) |
| CA19-9 (U/mL) | | |
| ≤median | 11 | (28%) |
| >median | 10 | (26%) |
| Unknown | 18 | (46%) |
| Performance status, n (%) | | |
| 0 + 1 | 30 | (77%) |
| 2 + 3 | 5 | (13%) |
| Unknown | 4 | (10%) |

| | |
|---|---|
| The Charlson age comorbidity index | unknown |

B)

| Clinical variables | Study population (n = 686) | |
|---|---|---|
| Age, (years) | | |
| Median (min, max) | 67 | (37-89) |
| Gender, n (%) | | |
| Male | 349 | (51%) |
| Female | 337 | (49%) |
| Number of metastatic sites, n (%) | | |
| ≤1 site | 620 | (90%) |
| >1 site | 66 | (10%) |
| Liver metastasis, n (%) | | |
| Only liver | 224 | (33%) |
| Other | 462 | (67%) |
| BMI | | |
| Median (min, max) | 23 | (14-39) |
| Stage | | |
| 1 | 13 | (2%) |
| 2 | 103 | (15%) |
| 3 | 198 | (29%) |
| 4 | 369 | (54%) |
| unknown | 3 | (<1%) |
| Diabetes | | |
| Yes | 157 | (23%) |
| No | 523 | (76%) |
| Unknown | 6 | (<1%) |
| Tobacco | | |
| Ever | 409 | (60%) |
| Never | 209 | (30%) |
| Unknown | 68 | (10%) |
| Alcohol | | |
| <DHAR | 473 | (69%) |
| >DHAR | 144 | (21%) |
| Unknown | 69 | (10%) |
| CA19-9 (U/mL) | | |
| ≤median | 251 | (37%) |
| >median | 245 | (35%) |
| Unknown | 190 | (28%) |
| Performance status, n (%) | | |
| 0 + 1 | 538 | (79%) |
| 2 + 3 | 78 | (11%) |
| unknown | 70 | (10%) |
| The Charlson age comorbidity index | | |
| <4 | 458 | (67%) |
| ≥4 | 218 | (32%) |
| Unknown | 10 | (1%) |

Patients with Metastatic Melanoma

PRO-C11-511 levels were measured in pretreatment serum samples from 35 patients with metastatic melanoma treated with anti-PD-1 therapy (pembrolizumab). The patients were treated with pembrolizumab as the standard of care at Copenhagen University Hospital, Herlev, after informed consent and approval by the Ethics Committee for the Capital Region of Denmark in compliance with the Helsinki Declaration 1975. Serum samples were measured blinded.

Patients with Various Cancer Types

Pro-C11-511 levels were measured in a samples from a further patient cohort consisting of 222 cancer samples and 33 healthy samples. It included 20 patients each of pancreatic-, colorectal-, kidney-, stomach-, breast-, bladder-, lung-, melanoma-, head and neck- and prostate-cancer, 19 ovarian cancer patients, 3 liver cancer patients and 33 age matched healthy controls. All cancer samples were obtained from Proteogenex (Los Angeles, CA, USA) and the healthy controls were obtained from BioIVT (Westbury, NY, USA). A summary of the cohort characteristics can be found in Table 3.

TABLE 3
Patient demographics of the cohort.

| | Healthy | | Cancer | | Total | |
|---|---|---|---|---|---|---|
| | (N = 33) | | (N = 222) | | (N = 255) | |
| Diagnosis | | | | | | |
| Healthy | 33 | (100%) | 0 | (0%) | 33 | (12.9%) |
| Bladder cancer | 0 | (0%) | 20 | (9.0%) | 20 | (7.8%) |
| Breast cancer | 0 | (0%) | 20 | (9.0%) | 20 | (7.8%) |
| Colorectal cancer | 0 | (0%) | 20 | (9.0%) | 20 | (7.8%) |
| Head & neck | 0 | (0%) | 20 | (9.0%) | 20 | (7.8%) |
| cancer | | | | | | |
| Kidney cancer | 0 | (0%) | 20 | (9.0%) | 20 | (7.8%) |
| Liver cancer | 0 | (0%) | 3 | (1.4%) | 3 | (1.2%) |
| Lung cancer | 0 | (0%) | 20 | (9.0%) | 20 | (7.8%) |
| Melanoma | 0 | (0%) | 20 | (9.0%) | 20 | (7.8%) |
| Ovarian cancer | 0 | (0%) | 19 | (8.6%) | 19 | (7.5%) |
| Pancreatic cancer | 0 | (0%) | 20 | (9.0%) | 20 | (7.8%) |
| Prostate cancer | 0 | (0%) | 20 | (9.0%) | 20 | (7.8%) |
| Stomach cancer | 0 | (0%) | 20 | (9.0%) | 20 | (7.8%) |
| Stages | | | | | | |
| I | 0 | (0%) | 7 | (3.2%) | 7 | (2.7%) |
| II | 0 | (0%) | 49 | (22.1%) | 49 | (19.2%) |
| III | 0 | (0%) | 93 | (41.9%) | 93 | (36.5%) |
| IV | 0 | (0%) | 73 | (32.9%) | 73 | (28.6%) |
| Missing | 33 | (100%) | 0 | (0%) | 33 | (12.9%) |
| Age (years) | | | | | | |
| Mean (SD) | 57.7 | (5.69) | 59.3 | (11.2) | 59.1 | (10.7) |
| Median | 57.0 | | 61.0 | | 60.5 | |
| [Min, Max] | [49.0, 69.0] | | [30.0, 87.0] | | [30.0, 87.0] | |
| Missing | 0 | (0%) | 1 | (0.5%) | 1 | (0.4%) |
| Sex | | | | | | |
| Male | 21 | (63.6%) | 121 | (54.5%) | 142 | (55.7%) |
| Female | 12 | (36.4%) | 101 | (45.5%) | 113 | (44.3%) |
| Ethnicity | | | | | | |
| Black | 13 | (39.4%) | 0 | (0%) | 13 | (5.1%) |
| Caucasian | 11 | (33.3%) | 222 | (100%) | 233 | (91.4%) |
| Hispanic | 9 | (27.3%) | 0 | (0%) | 9 | (3.5%) |
| BMI | | | | | | |
| Mean (SD) | NA | (NA) | 26.4 | (4.28) | 26.4 | (4.28) |
| Median | NA | | 25.8 | | 25.8 | |
| [Min, Max] | [NA, NA] | | [17.9, 41.4] | | [17.9, 41.4] | |
| Missing | 33 | (100%) | 1 | (0.5%) | 34 | (13.3%) |

### Statistical Analysis

Biomarker results are reported in accordance with the REMARK (reporting recommendations for tumor marker prognostic study) guidelines.

A Kruskal-Wallis test was used to test the difference between PRO-C11-253 and PRO-C1-511 biomarker levels of PC, CP and healthy controls. Kaplan-Meier curves were used to assess the difference between high (>75% quartile) and low (<75% quartile) PRO-C11-253 and PRO-0511 biomarker levels and overall survival (OS). Patients were followed from the date of inclusion in the BIOPAC study to the end of follow up or until death from any cause, whichever came first. A univariate Cox proportional-hazard regression model was to calculate the hazard ratios (HR) with 95% confidence interval (CI) for the OS per PRO-C11-511 biomarker levels and clinical co-variates: age (continuous), gender (female vs. male), number of metastatic sites (<1 vs. ≤1), liver metastasis (yes vs. no/other), BMI (continuous), stage (continuous and III+IV vs. I+II), diabetes (yes vs. no), tobacco use (ever vs. never), alcohol use (below the Danish Health Authority recommendations vs. abuse), CA19-9 (>median), performance status (PS) (2 vs. ≤1, 2 vs. 0, 2+3 vs. 0+1) and The Charlson age cormibidity index (CACI) (≥4 vs. <4) (Asano et al., 2017). A multivariate Cox proportional-hazard regression model including PRO-C11-511, age, metastatic sites (<1 vs. ≤1), liver metastasis (yes vs. no/other), stage (III+IV vs. I+II), CA19-9 (>median) and PS (2+3 vs. 0+1) was used to evaluate potential independent prognostic value of the PRO-C11-511 biomarker. A p-value of p<0.05 was considered statistically significant. Graph design and statistical analyses were performed using GraphPad Prism Version 8.2 (GraphPad Software, Inc.) and MedCalc version 19.3 (Medcalc Software).

The association between PRO-C11-511 levels and progression-free survival (PFS) and overall survival (OS) in patients with metastatic melanomas were assessed by Kaplan Meier analyses and Cox regression analyses, alone and after adjusting for tumor PDL1 expression and BRAF mutational status.

The analysis of the samples acquired from patients with various cancer types involved comparisons of PRO-C11-511 levels across groups using ordinary one-way ANOVA followed by pair-wise comparisons to the control group using the Dunnett test. A p-value below 0.05 was considered significant. Statistical analysis and graphs were done in R version 4.0.4 (R Core Team (2021), R Foundation for Statistical Computing, Vienna, Austria, https://www.R-project.org).

### Results

### Specificity of PRO-C11-253 and PRO-C11-511 Antibodies

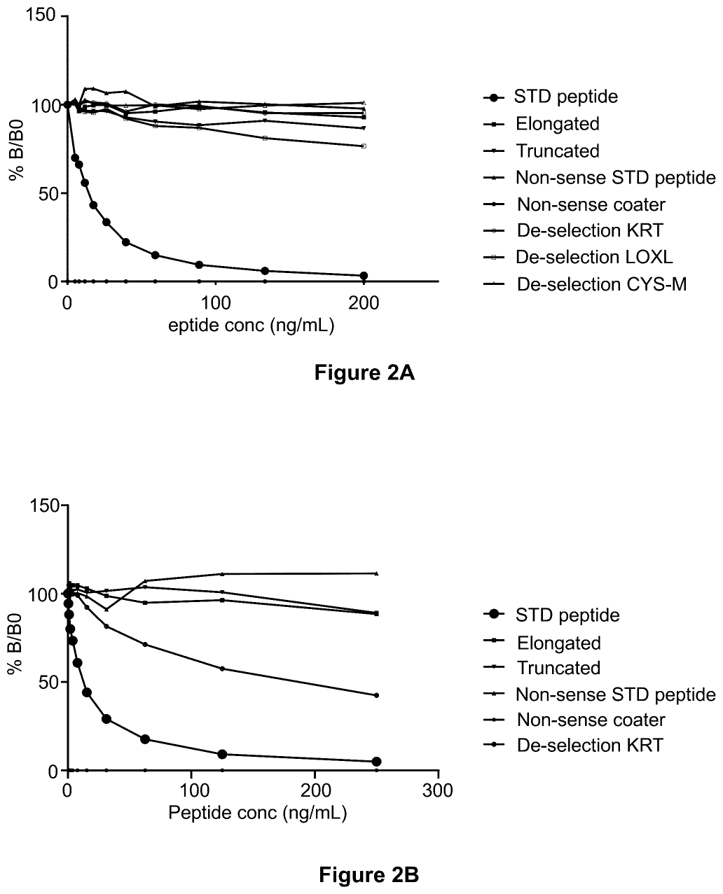

To ensure antibody specificity of the PRO-C11-253 and PRO-C11-511 antibodies against their respective binding sites, their reactivity was tested against a standard, elongated, truncated, and non-sense standard peptide in a preliminary competitive ELISA, as discussed above. The non-sense peptide chosen for PRO-C11-511 antibody was the PRO-C11-253 standard peptide (see table 1 above). Furthermore, their reactivity was tested against de-selection peptides with mismatch amino acid sequences. Both selection peptides inhibited the signal in a dose-dependent manner with their respective antibodies. There was no reactivity against the elongated, truncated peptides and non-sense peptides, and minimal reactivity against the de-selection peptides. Furthermore, there was no detectable signal observed when using the non-sense biotinylated coating peptide (FIG. 2).

Together, these data suggest that the monoclonal antibodies exhibit high neo-epitope specificity towards their respective target sites.

### Technical Evaluation of PRO-C11-253 and PRO-C11-511 ELISAs

The technical performance of the PRO-C11-253 and PRO-C11-511 ELISA assays are summarized in table 4. The measuring range (LLMR to ULMR) of the assay was determined to 3.1-103.0 ng/mL for PRO-C11-253 and 1.6-117.5 ng/mL for PRO-C11-511. The intra- and inter-assay variation for PRO-C11-253 was 5% and 9% and for PRO-C11-511% and 5%, respectively. The mean dilution recovery for human serum was 112% for PRO-C11-253 and 85.5% for PRO-C11-511 observed from undiluted to a 1:2 dilution. Mean spiking recovery for was determined to 102.7% for PRO-C11-253 and 92.5% for PRO-C11-511. The analyte stability was analyzed according to freeze/thaw cycles and sample stability at 4° C. and 20° C. The analyte recovery in serum was 99.9% for PRO-C11-253 and 93.0% for PRO-C11-511 after 4 freeze/thaw cycles. The analytes were recovered after prolonged storage of human serum at 4° C. for 2 hours to 48 hours, resulting in a recovery ranging from 105.2-92.2% for PRO-C11-253 and 91.5-90% for PRO-C11-511. For 20° C., 2 hours to 48 hours, the recovery range for PRO-C11-253 was 104.7-80.1% and for PRO-C11-511 91.2-119.1%. These data indicate that the serum analytes PRO-C11-253 and PRO-C11-511 are binding to are stable at 4° C. and 20° C. for up to 48 hours. No interference was detected from either low or high contents of lipids or hemoglobin with recoveries ranging from 83.5-127.7% for PRO-C11-253 and 95.6 to 107.9% for PRO-C11-511.

TABLE 4
Technical validation of PRO-C11-C253 and PRO-C11-511 assays

| Technical validation step | PRO-C11-253 | PRO-C11-511 |
|---|---|---|
| Detection Range | 3.11 ng/ml-103 ng/ml | 1.6-117.5 ng/ml |
| Intra-assay variation | 5% | 9.0% |
| Inter-assay variation | 9% | 5.% |
| Dilution recovery in serum | 112% | 85.5% |
| Freeze-thaw recovery in | 99.9% | 93.0% |
| serum | | |
| Spiking Recovery | 102.7% | 92.5% |
| Analyte stability range in | 105.2-92.2% | 91.5-90.0% |
| serum 4° C., 2 h-48 h | | |
| Analyte stability range in | 104.7-80.1% | 91.2-119.1% |
| serum 20° C., 2 h-48 h | | |
| Interference: | | |
| Recovery in biotin, low/high | 91.9/95.2% | 99.1/99.9% |
| Recovery in lipid, low/high | 83.5/88.1% | 103.7/107.9% |
| Recovery in haemoglobin, | 106.6/124.7% | 95.6/97.0% |
| low/high | | |

Clinical Evaluation of PRO-C11-253 and PRO-C11-511 in Serum from Healthy Donors, Patients with CP and Patients with PC

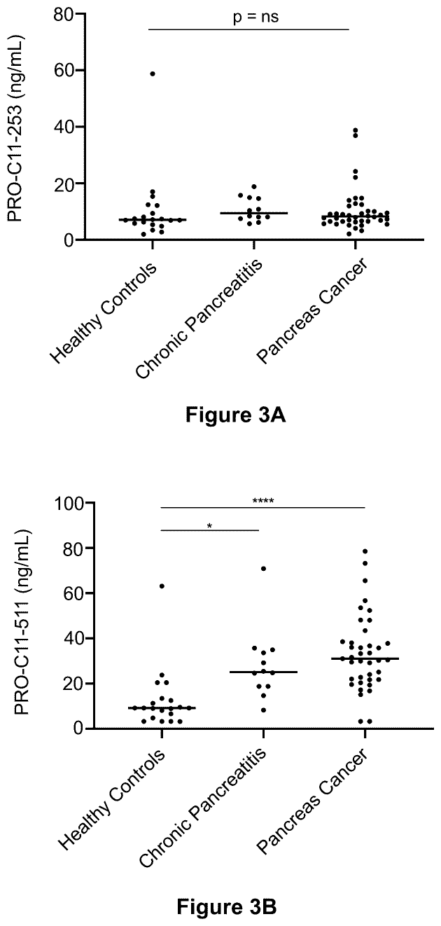

To evaluate the clinical relevance of PRO-C11-253 and PRO-C11-511 the biomarkers were measured in serum from a cohort of healthy controls (n=20), patients with CP (n=12) and patients with PC (n=39). There was no significant difference in PRO-C11-253 between healthy controls, CP and PC patients, whereas, PRO-C11-511 was significantly increased in patients with CP (p=0.0116) and PC (p<0.0001) compared to healthy controls (FIGS. 3A and B). There was no difference between biomarker levels and stage of PC (data not shown). Furthermore, there was no correlation between PRO-C11-253 and PRO-C11-511 biomarker measurements (r, 0.14, p=0.3827).

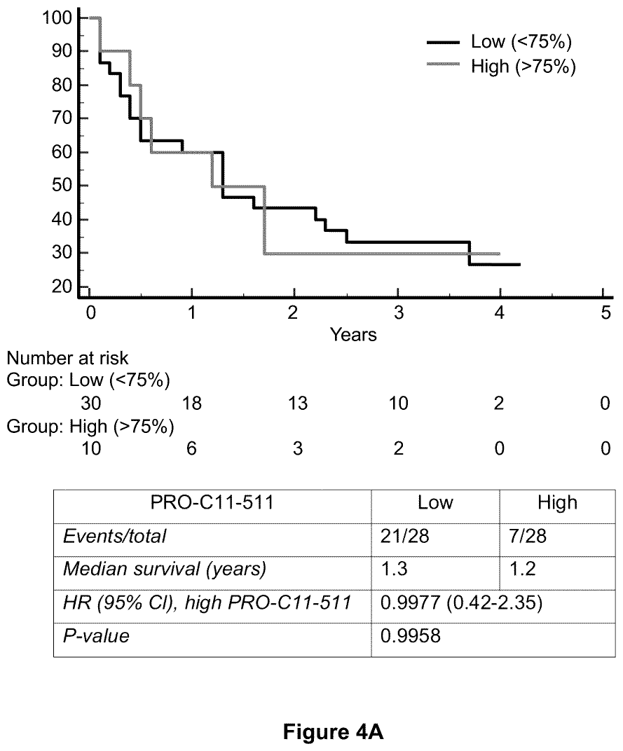

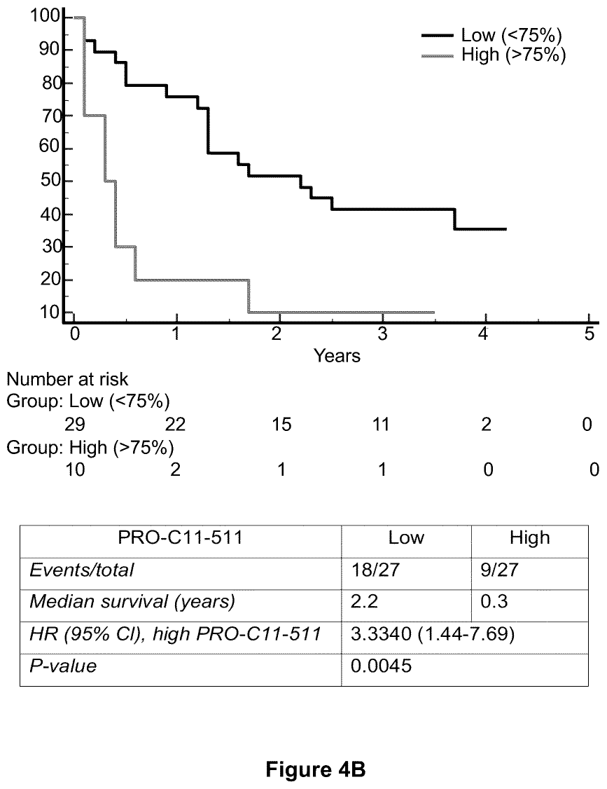

Next, the prognostic value of PRO-C11-253 and PRO-C11-511 was investigated using Kaplan-Meier curves and Cox-proportional hazard models in the same validation cohort of 39 PC patients as mentioned above. Kaplan-Meier curves and Cox-proportional hazard models showed that high (>75%) and low (<75%) biomarker levels of PRO-C11-253 were not associated with OS (p=0.9958, HR 1.00, 95% CI 0.42-2.35). Median OS for patients with high (>75%) and low (<75%) PRO-C11-253 were 1.2 years and 1.3 years, respectively (FIG. 4A). Interestingly, when evaluating the association between PRO-C11-511 biomarker levels and survival, high PRO-C11-511 (>75%) was significantly associated with shorter OS compared to low PRO-C11-511 (<75%) (p=0.0045, HR 3.33, 95% CI 1.44-7.69). Furthermore, there was a 7-fold difference between the median OS for patients with high (>7%: 0.3 years) and low PRO-C11-511 (<75%: 2.2 years) (FIG. 4B). These data indicate that PRO-C11-511, but not PRO-C11-253, could have prognostic potential in patients with PC and that measuring specific epitopes on the same protein may provide different value.

### Confirmation of PRO-C11-511 as a Prognostic Biomarker in a Validation Cohort

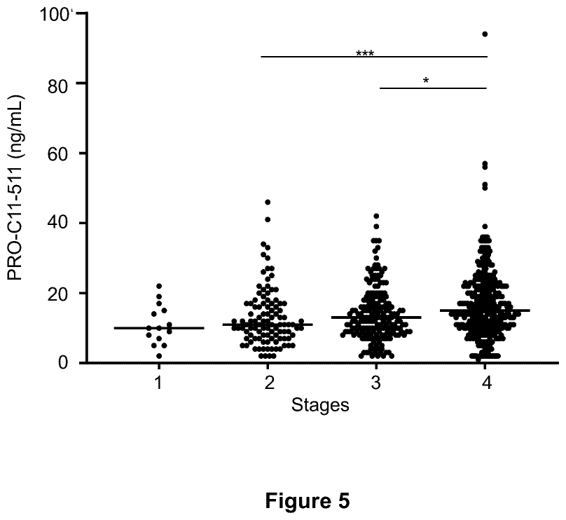

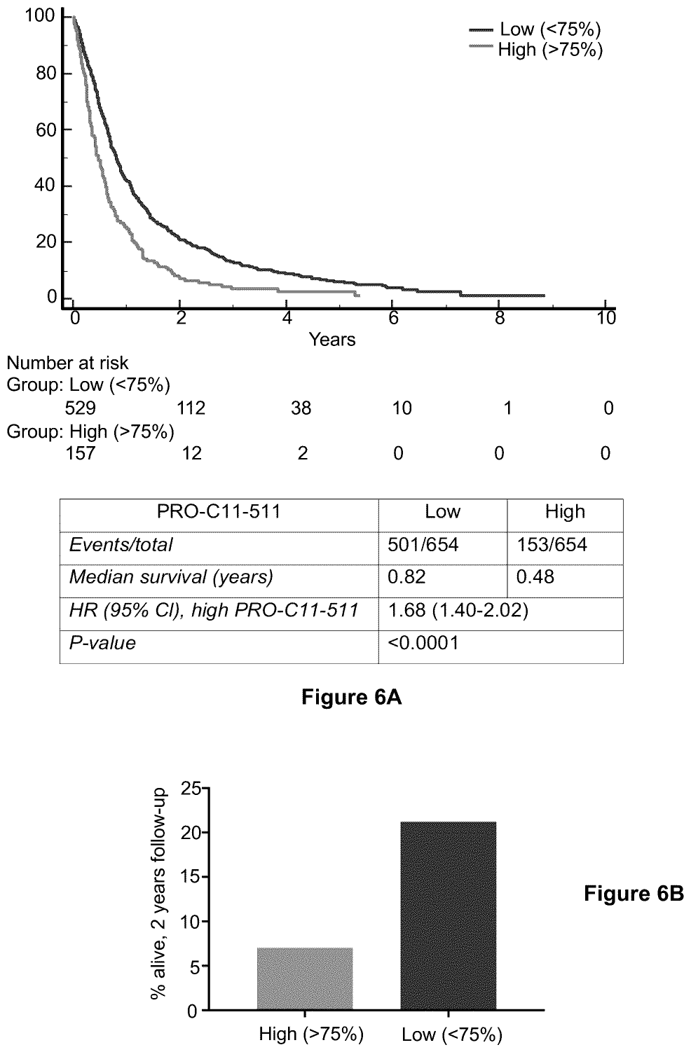

To validate the prognostic biomarker potential of PRO-C11-511 found in the PC discovery cohort described above PRO-C11-511 levels were measured in larger cohort of patients with PC (n=686). Interestingly, when dividing patients into their respective disease stage (stage I-IV), there was a significant increase in PRO-C11-511 from stage II-IV and stage III to IV (FIG. 5). Next, we looked at the association between high (>75%) and low (<75%) PRO-C11-511 and OS. Like with discovery cohort, high PRO-C11-511 (>75%) was significantly associated with shorter OS in the larger study population compared to low PRO-C11-511 (<7%) (p<0.0001, HR 1.68, 95% CI 1.40-2.02). Median OS for patients with high PRO-C11-511 (>75%) was 0.48 years and 0.82 years for patients with low PRO-C11-511 (<75%) (FIG. 6A). Interestingly, 2 years post-baseline there was only 7.0% alive in the high PRO-C11-511 patient group compared to 21% in the low PRO-C11-511 patient group (FIG. 6B). These data confirm that PRO-C11-511 has prognostic potential in patients with PC.

To evaluate if the association of PRO-C11-511 and OS was independent of clinical co-variates a multivariate Cox analysis was performed adjusting PRO-C11-511 for age, metastatic sites (<1 vs. ≤1), liver mets (yes vs. no/other), stage (III+IV vs. I+II), CA19-9 (>median) and PS (2+3 vs. 0+1). The analysis showed that high levels of PRO-C11-511 (>75%) were not dependent on the clinical co-variates and therefore still predictive of OS in PC patients after adjustment (p<0.0021, HR 1.41, 95% CI 1.13-1.74 (table 5A and B).

TABLE 5
Association between biomarker levels, clinical covariates and outcome for
patients with pancreatic cancer. Uni- (a) and multivariate (b) cox proportional-hazards
regression were used to calculate the hazard ratios (HR) with 95% Cl and p-values.
P < 0.05 is considered significant. DHAR: Danish Health Authority recommendations, PS:
pre-treatment performance status, CACl: the Charlson age comorbidity index.
A)
UNIVARIATE ANALYSIS

| | | Overall survival | | |
|---|---|---|---|---|
| Variables | Univariate analysis | HR | 95% Cl | P-value |
| PRO-C11 | Continuous | 1.0201 | 1.0123 to 1.0279 | <0.0001 |
| | <75% vs. >75% | 1.68 | 1.40-2.02 | <0.0001 |
| Age | Per year increase | 1.0110 | 1.0028-1.0193 | 0.0082 |
| Gender | Female vs. male | 0.9893 | 0.8610 to 1.1369 | 0.8799 |
| Number of | Continuous | 1.7451 | 1.5989 to 1.9046 | <0.0001 |
| metastatic | | | | |
| sites | | | | |
| | >1 vs. ≤1 | 1.7588 | 1.4018 to 2.2068 | <0.0001 |
| Liver | Only liver vs. other | 2.22 | 1.90-2.59 | <0.0001 |
| metastasis | | | | |
| BMI | Continuous | 0.9894 | 0.9717 to 1.0074 | 0.2450 |
| Stage | Continuous | 1.3415 | 1.2762 to 1.4102 | <0.0001 |
| | 1 + 2 vs. 3 + 4 | 2.87 | 2.34-3.53 | <0.0001 |
| Diabetes | Yes vs. no | 1.0893 | 0.9280 to 1.2787 | 0.2955 |
| Tobacco | Ever vs. never | 1.0695 | 0.9171-1.2474 | 0.3915 |
| Alcohol | >DHAR vs. <DHAR | 0.999 | 0.8460-1.1819 | 0.9992 |
| CA19-9 | >median va. ≤median | 2.0329 | 1.7174-2.4064 | <0.0001 |
| PS | Continuous | 1.3585 | 1.2695-1.4536 | <0.0001 |
| | 2 + 3 vs. 0 + 1 | 2.19 | 1.77-2.71 | <0.0001 |
| CACl | High (≥4 vs. < 4) | 1.14 | 0.98-1.31 | 0.0838 |

B)
MULTIVARIATE ANALYSIS

| | | Overall survival | | |
|---|---|---|---|---|
| Variables | Multivariate analysis | HR | 95% Cl | P-value |
| PRO-C11 | >75% vs. ≤75% | 1.41 | 1.13-1.74 | 0.0021 |
| Age | Continuous | 1.01 | 1.00-1.02 | 0.0708 |
| Metastatic | >1 vs. ≤1 | 1.11 | 0.78-1.57 | 0.5636 |
| sites | | | | |
| Liver mets | Only liver vs. other | 1.49 | 1.20-1.84 | 0.0003 |
| Stage | 3 + 4 vs. 1 + 2 | 2.15 | 1.50-3.10 | <0.0001 |
| CA19-9 | >median vs ≤median | 1.68 | 1.36-2.06 | <0.0001 |
| PS | 2 + 3 vs. 0 + 1 | 2.06 | 1.60-2.71 | <0.0001 |

### Clinical Evaluation of PRO-C11-511 in Metastatic Melanoma Patients

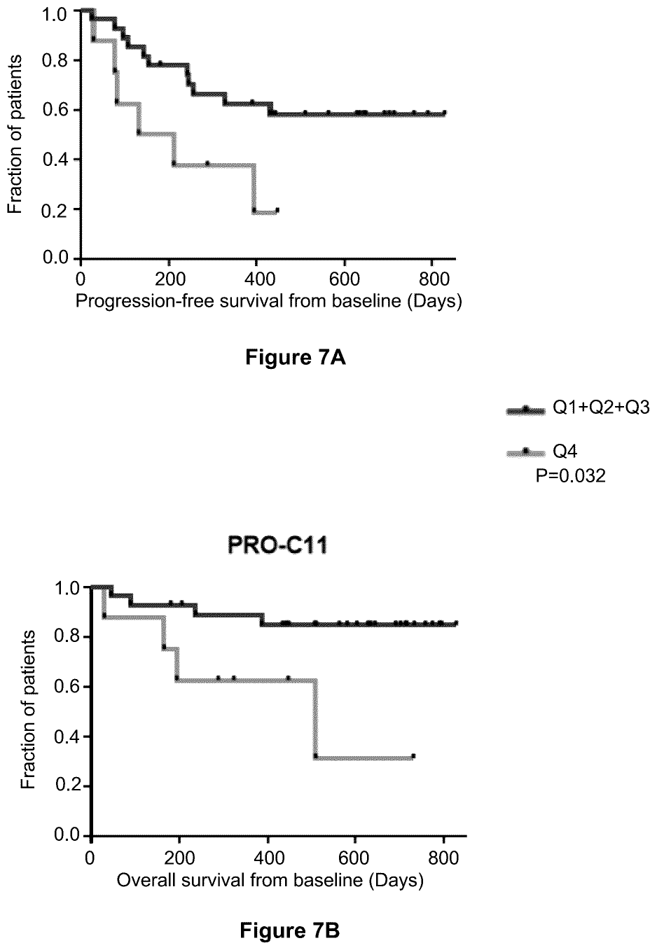

The association between PRO-C11-511 and survival outcomes in the metastatic melanoma patients was evaluated by Kaplan-Meier analysis. Using the 75th percentile cut point, it was found that patients with high PRO-C11-511 levels (>75th percentile) had significantly worse PFS (p=0.032) and OS (p=0.020) (FIG. 7). In support, univariate Cox regression identified high (>75th percentile) baseline PRO-C11-511 as predictor of poor PFS (HR=2.87, 95% CI=1.05-7.87, p=0.040) and OS (HR=4.58, 95% CI=1.13-18.65, p=0.034). Moreover, when PRO-C11-511 was adjusted for PDL1 expression (≥1%) and BRAF mutations, the biomarker remained independently predictive of poor PFS (HR=3.66, 95% CI=1.22-10.98, p=0.021) and OS (HR=7.85, 95% CI=1.32-46.78, p=0.024) (table 6).

TABLE 6
Cox regression analysis for predicting progression-free survival and overall survival outcome

| | Progression-free survival | | | Overall survival | | |
|---|---|---|---|---|---|---|
| Univariate analysis | HR | 95% CI | p-value | HR | 95% CI | p-value |
| PRO-C11-511, Q4 vs | 2.87 | 1.05-7.87 | 0.040 | 4.58 | 1.13-18.65 | 0.034 |
| Q1 + Q2 + Q3 | | | | | | |

| | Multivariate analysis adjusted for PDL1 expression (≥1%) and BRAF mutations | | | | | |
|---|---|---|---|---|---|---|
| | HR | 95% CI | p-value | HR | 95% CI | p-value |
| PRO-C11-511, Q4 vs | 3.66 | 1.22-10.98 | 0.021 | 7.85 | 1.32-46.78 | 0.024 |
| Q1 + Q2 + Q3 | | | | | | |

Clinical Evaluation of Pro-C11-511 in Patients with Various Cancer Types

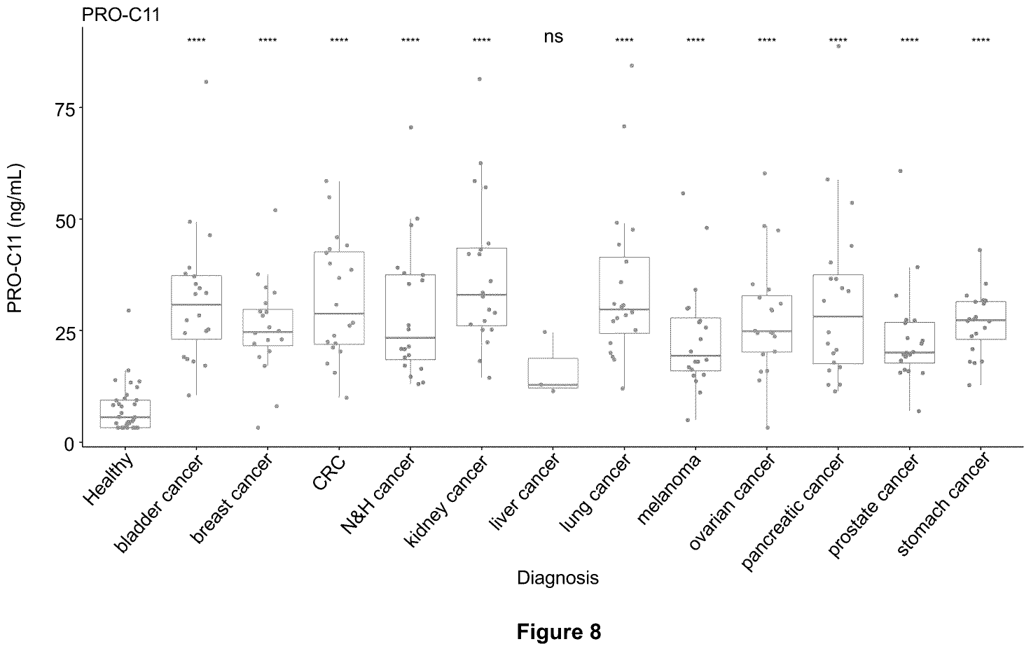

To confirm the clinical relevance of PRO-C11-511 in other types of cancer the levels of Pro-C11-511 were measured in serums samples from a further patient cohort consisting of 222 cancer samples (20 patients each of pancreatic-, colorectal-, kidney-, stomach-, breast-, bladder-, lung-, melanoma-, head and neck- and prostate-cancer, 19 ovarian cancer patients, 3 liver cancer patients) and 33 age matched healthy controls. PRO-C11-511 levels were elevated in all cancers types, and were significantly elevated in all cancer types except liver cancer, compared to the healthy controls (FIG. 8).

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used to mean ‘including or consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

Asano, T. et al. (2017) ‘The Charlson age comorbidity index predicts prognosis in patients with resected pancreatic cancer’, International Journal of Surgery. doi: 10.1016/j.ijsu.2017.01.115.

Bager, C. L. et al. (2015) ‘Collagen degradation products measured in serum can separate ovarian and breast cancer patients from healthy controls: A preliminary study’, Cancer Biomarkers. doi: 10.3233/CBM-150520.

Bray, F. et al. (2018) ‘Global cancer statistics 2018: GLOBOCAN estimates of incidence and mortality worldwide for 36 cancers in 185 countries’, CA: A Cancer Journal for Clinicians. doi: 10.3322/caac.21492.

Chen, I. M. et al. (2020) ‘Clinical value of serum hyaluronan and propeptide of type III collagen in patients with pancreatic cancer’, International Journal of Cancer, 146(10), pp. 2913-2922. doi: 10.1002/ijc.32751.

Combet, C. et al. (2000) ‘NPS@: Network Protein Sequence Analysis’, Trends in Biochemical Sciences, 25(3), pp. 147-50. doi: 10.1016/s0968-0004(99)01540-6.

Franco, O. E. et al. (2010) ‘Cancer associated fibroblasts in cancer pathogenesis’, Seminars in Cell and Developmental Biology, pp. 33-39. doi: 10.1016/j.semcdb.2009.10.010.

García-Pravia, C. et al. (2013) ‘Overexpression of COL11A1 by Cancer-Associated Fibroblasts: Clinical Relevance of a Stromal Marker in Pancreatic Cancer’, PLoS ONE, 8(10), pp. 1-13. doi: 10.1371/journal.pone.0078327.

Gefter, M. L., Margulies, D. H. and Scharff, M. D. (1977) ‘A simple method for polyethylene glycol-promoted hybridization of mouse myeloma cells’, Somatic Cell Genetics, 3(2), pp. 231-236. doi: 10.1007/BF01551818.

Hosein, A. N., Brekken, R. A. and Maitra, A. (2020) ‘Pancreatic cancer stroma: an update on therapeutic targeting strategies’, Nature Reviews Gastroenterology and Hepatology. doi: 10.1038/s41575-020-0300-1.

Jensen, C. et al. (2018) ‘Altered type III collagen turnover measured in pre-treatment serum predicts outcome in metastatic melanoma patients treated with Ipilimumab’, European Journal of Cancer, 92.

Kalluri, R. and Zeisberg, M. (2006) ‘Fibroblasts in cancer’, Nature Reviews Cancer, 6(5), pp. 392-401. doi: 10.1038/nrc1877.

Kang, C. Y. et al. (2014) ‘Clinical Significance of Serum COL6A3 in Pancreatic Ductal Adenocarcinoma’, Journal of Gastrointestinal Surgery, 18(1), pp. 7-15. doi: 10.1007/s11605-013-2326-y.

Kehlet, S. N. et al. (2016) ‘Excessive collagen turnover products are released during colorectal cancer progression and elevated in serum from metastatic colorectal cancer patients’, Scientific Reports. Nature Publishing Group, 6(July), pp. 1-7. doi: 10.1038/srep30599.

McGuigan, A. et al. (2018) ‘Pancreatic cancer: A review of clinical diagnosis, epidemiology, treatment and outcomes’, World Journal of Gastroenterology. doi: 10.3748/wjg.v24.i43.4846.

Nissen, N. I., Karsdal, M. and Willumsen, N. (2019) ‘Collagens and Cancer associated fibroblasts in the reactive stroma and its relation to Cancer biology’, Journal of Experimental & Clinical Cancer Research, 38(1), p. 115. doi: 10.1186/s13046-019-1110-6.

Raglow, Z. and Thomas, S. M. (2015) ‘Tumor matrix protein collagen XIα1 in cancer’, Cancer Letters. doi: 10.1016/j.canlet.2014.12.011.

Rousseau, J. et al. (1996) ‘Processing of Type XI Collagen rich protein region encoded by exon I (Zhidkova , N . I .’, 271(39), pp. 23743-23748.

Vázquez-Villa, F. et al. (2015) ‘COL11A1/(pro)collagen 11A1 expression is a remarkable biomarker of human invasive carcinoma-associated stromal cells and carcinoma progression’, Tumor Biology, pp. 2213-2222. doi: 10.1007/s13277-015-3295-4.

Wang, S. et al. (2018) ‘Extracellular matrix (ECM) circulating peptide biomarkers as potential predictors of survival in patients (pts) with untreated metastatic pancreatic ductal adenocarcinoma (mPDA) receiving pegvorhyaluronidase alfa (PEGPH20), nab-paclitaxel (A), and gemcita’, Journal of Clinical Oncology, 15, pp. 12030-12030. doi: 10.1200/JC0.2018.36.15_supp1.12030.

Willumsen, N. et al. (2013) ‘Extracellular matrix specific protein fingerprints measured in serum can seperate pancreatic cancer patients from healthy controls’, BMC cancer, 13, p. 554. doi: 10.1186/1471-2407-13-554.

Willumsen, N. et al. (2014) ‘Serum biomarkers reflecting specific tumor tissue remodeling processes are valuable diagnostic tools for lung cancer’, Cancer Medicine, 3(5), pp. 1136-1145. doi: 10.1002/cam4.303.

World Health Organization: Latest global cancer data (2018). Available at: https://www.who.int/cancer/PRGlobocanFinal.pdf.

Yoshioka, H. and Ramirez, F. (1990) ‘Pro-a 1 (XI) Collagen’, J Biol Chem, 265(11), pp. 6423-6426

1: A monoclonal antibody that specifically recognises and binds to the C-terminus of a peptide having the C-terminus amino acid sequence DGSKGPTISA (SEQ ID NO: 1).

2: The monoclonal antibody of claim 1, wherein the monoclonal antibody does not specifically bind to a peptide having the C-terminus amino acid sequence DGSKGPTISAQ (SEQ ID NO: 2).

3: The monoclonal antibody of claim 1, wherein the monoclonal antibody does not specifically bind to a peptide having the C-terminus amino acid sequence DGSKGPTIS (SEQ ID NO: 3).

4: The monoclonal antibody of claim 1, wherein the monoclonal antibody is raised against a synthetic peptide having the C-terminus amino acid sequence DGSKGPTISA (SEQ ID NO: 1).

5: A method of immunoassay, the method comprising:
i) contacting a sample from a patient with a monoclonal antibody that specifically recognises and binds to the C-terminus of a peptide having the C-terminus amino acid sequence DGSKGPTISA (SEQ ID NO: 1); and
ii) detecting and determining the amount of binding between said monoclonal antibody and peptides in the sample.

6: The method of claim 5, wherein the method is a method of immunoassay for detecting and/or monitoring a disease in a patient and/or assessing the severity or prognosis of a disease in a patient, the method further comprising:
iii) correlating said amount of binding of said monoclonal antibody as determined in step (ii) with values associated with normal healthy subjects, and/or with values associated with known disease severity or prognosis, and/or with values obtained from said patient at a previous time point, and/or with a predetermined cut-off value.

7: The method of claim 6, wherein the disease is pancreatic cancer or chronic pancreatitis.

8: The method of claim 6, wherein the disease is melanoma.

9: The method of claim 6, wherein the disease is a cancer with a stroma- and cancer associated fibroblast-rich tumor micro environment.

10: The method of claim 6, wherein the disease is bladder cancer, breast cancer, colorectal cancer, head and neck cancer, kidney cancer, liver cancer, lung cancer, melanoma, ovarian cancer, pancreatic cancer, prostate cancer, or stomach cancer.

11: The method of claim 6, wherein the disease is a cancer, and the method is a method for assessing a likely period of patient survival or progression-free survival with treatment with one or more chemotherapeutic agents and/or immune checkpoint inhibitors.

12: The method of claim 5, wherein the monoclonal antibody does not specifically bind to a peptide having the C-terminus amino acid sequence DGSKGPTISAQ (SEQ ID NO: 2).

13: The method of claim 5, wherein the monoclonal antibody does not specifically bind to a peptide having the C-terminus amino acid sequence DGSKGPTIS (SEQ ID NO: 3).

14: The method of claim 5, wherein the monoclonal antibody is raised against a synthetic peptide having the C-terminus amino acid sequence DGSKGPTISA (SEQ ID NO: 1).

15: The method of claim 5, wherein the sample is a biofluid sample selected from blood, serum or plasma.

16: The method of claim 5, wherein the immunoassay is a competition assay or a sandwich assay.

17: The method of claim 5, wherein the immunoassay is a radio-immunoassay or an enzyme-linked immunosorbent assay.

18: An immunoassay kit comprising a monoclonal antibody that specifically recognises and binds to the C-terminus of a peptide having the C-terminus amino acid sequence DGSKGPTISA (SEQ ID NO: 1), and at least one of;
a streptavidin coated well plate;
a biotinylated peptide Biotin-L-DGSKGPTISA (SEQ ID NO: 4), wherein L is an optional linker;
a secondary antibody for use in a sandwich immunoassay;
a calibrator protein comprising the sequence DGSKGPTISA (SEQ ID NO: 1);
an antibody biotinylation kit;
an antibody HRP labelling kit;
an antibody radiolabelling kit; or
an assay visualisation kit.

19: The immunoassay kit of claim 18, wherein the monoclonal antibody does not specifically bind to a peptide having the C-terminus amino acid sequence DGSKGPTISAQ (SEQ ID NO: 2).

20: The immunoassay kit of claim 18, wherein the monoclonal antibody does not specifically bind to a peptide having the C-terminus amino acid sequence DGSKGPTIS (SEQ ID NO: 3).

21: The immunoassay kit of claim 18, wherein the monoclonal antibody is raised against a synthetic peptide having the C-terminus amino acid sequence DGSKGPTISA (SEQ ID NO: 1).


SEQUENCE LISTING (16 sequences)

SEQ ID NO: 1 - protein, 10 aa, Artificial Sequence
  502..511: PRO-C11-511 peptide C-terminus target sequence of type XI collagen used to raise a monoclonal antibody
DGSKGPTISA

SEQ ID NO: 2 - protein, 11 aa, Artificial Sequence
  Extended PRO-C11-511 peptide C-terminus target sequence
DGSKGPTISAQ

SEQ ID NO: 3 - protein, 9 aa, Artificial Sequence
  Truncated PRO-C11-511 peptide C-terminus target sequence
DGSKGPTIS

SEQ ID NO: 4 - protein, 10 aa, Artificial Sequence
  Biotinylated target peptide
  SITE 1: Biotinylated via an optional linker
DGSKGPTISA

SEQ ID NO: 5 - protein, 10 aa, Artificial Sequence
  244..253: PRO-C11-253 peptide target sequence of type XI collagen used to raise a monoclonal antibody
DSSAPKAAQA

SEQ ID NO: 6 - protein, 13 aa, Artificial Sequence
  Immunogenic peptide for PRO-C11-511 peptide
  SITE 1: Covalently cross-linked to Keyhole Limpet Hemocyanin
CGGDSSAPKAAQA

SEQ ID NO: 7 - protein, 13 aa, Artificial Sequence
  Immunogenic peptide for PRO-C11-253 peptide
  SITE 1: Covalently cross-linked to Keyhole Limpet Hemocyanin
CGGDGSKGPTISA

SEQ ID NO: 8 - protein, 10 aa, Artificial Sequence
  Biotinylated coating peptide
  SITE 1: Biotinylated
DSSAPKAAQA

SEQ ID NO: 9 - protein, 11 aa, Artificial Sequence
  Elongating coating peptide
DSSAPKAAQAQ

SEQ ID NO: 10 - protein, 9 aa, Artificial Sequence
  Truncated coating peptide
DSSAPKAAQ

SEQ ID NO: 11 - protein, 10 aa, Artificial Sequence
  Non-sense standard peptide
LLARDFEKNY

SEQ ID NO: 12 - protein, 11 aa, Artificial Sequence
  Non-sense coater peptide
  MOD_RES 11: Biotinylated
LLARDFEKNYK

SEQ ID NO: 13 - protein, 10 aa, Artificial Sequence
  Cystatin-M de-selection peptide
DPQVQKAAQA

SEQ ID NO: 14 - protein, 10 aa, Artificial Sequence
  Lysyl oxidase homolog 1 de-selection peptide
PDPGPEAAQA

SEQ ID NO: 15 - protein, 10 aa, Artificial Sequence
  Keratin-like protein KRT222 de-selection peptide
DEEALKAAQA

SEQ ID NO: 16 - protein, 10 aa, Artificial Sequence
  Mucin-4 de-selection peptide
VPQDAPTISA